(19) United States
(12) Patent Application Publication
(10) Pub. No.: US 2018/0132714 A1
(43) Pub. Date: May 17, 2018

(54) APPARATUS AND METHOD FOR MEASURING BRAIN PROCESSING SPEED

(71) Applicant: Infinite Mind, LC, Salt Lake City, UT (US)

(72) Inventors: Akihiro Kawamura, Ooamishirasato-Machi (JP); Jeffrey C. Flamm, Salt Lake City, UT (US)

(21) Appl. No.: 15/353,650

(22) Filed: Nov. 16, 2016

(51) Int. Cl. A61B 3/02; A61B 5/12; A61B 5/00

(52) U.S. Cl. CPC A61B 3/02; A61B 5/4064; A61B 5/123

(57) ABSTRACT

The present invention is directed to an apparatus for identifying the rate of frequency at which a person perceives distinct visual images or sounds. The device, product and method comprise means for presenting the image(s) or sounds, means for selecting the distinct image(s) or sounds, means for selecting an instance number, presenting the image or sound in an amount equal to the instance number, the distinct image being displayed on a screen, the sound being presented via a speaker, at a first frequency rate at which frequency the person in unable to perceive the correct number of images or sounds, means for decreasing the frequency rate presentation of the distinct image or sound to a second frequency rate, the second frequency rate being the frequency at which the person is able to perceive individual occurrences of the image or sound in an amount equal to the instance number, and capturing the value of the second display frequency. The captured display frequencies are instrumental in assessing and determining the rate or speed at which the tested person's brain perceives images or sound.

### BACKGROUND OF THE INVENTION

### 1. Field of the Invention

The present invention relates to apparatuses and methods for measuring and improving brain processing speed. In particular, the present invention relates to new systems and methods using rapid and decreasing rates in which visual images are displayed or auditory stimuli sounded in order to measure the speed at which the brain perceives the image or sound. In the case of visual images new and novel mechanisms of flicker fusion are used to identify brain processing rates. In the case of auditory stimuli new and novel mechanisms of sound presentation are used to measure when comprehension occurs.

### 2. Background and Related Art

While apparatuses and techniques currently exist that are used to measure the speed of brain processing challenges still exist including methods of preventing the participant from guessing which can cause unreliable testing data. Accordingly, it would be an improvement in the art to augment or even replace current techniques with the new techniques of the present invention to ensure the capture and assessment of reliable, accurate data used to measure the brain processing speed of participants.

For decades science has identified how the brain collects and processes visual information. It does so in windows of time known as windows of integration. If two or more visual images appear in a single window of integration the images appear as being coexistent. U.S. Pat. No. 9,095,295.

Scientists have long measured the brain's speed of information processing by using a flickering light whose frequency of flicker is altered. At lower frequencies the light appears to flicker. At higher frequencies the light appears to be steady without flicker. The point at which the flicker fuses to a perceived steady light is known as the critical flicker fusion threshold. Id.

The frequency at which critical flicker fusion threshold manifests to a particular person varies depending on the speed at which that person's brain processes visual images. This flicker fusion threshold has been used to measure ascending and descending thresholds. Ascending threshold occurs when the frequency of a flickering light starts low and is gradually increased until the light appears constant. Descending threshold occurs when the frequency of what appears to be a constant light starts out high and is gradually decreased until the light appears to flicker. Other applications of flicker testing include randomly presenting several different flicker frequencies or simultaneously presenting a steady image and a flickering image. These applications have been used to try to assess the participant's critical flicker fusion threshold. Id. Flicker applications have also employed a sequence of alternating positive and negative images at different frequencies in a stacked configuration of rows of images at different frequencies. Id.

As to measuring rates of auditory processing, a number of modalities have been used. For example, those skilled in the art have studied how sound is processed by the part of the brain known as the auditory cortex. Scientific researchers have taught that sound perception in the brain comprises a series of fast snapshots of sound and that the window of sound perception or comprehension in the brain is normally around 80 msec. For purposes of this application the term “window of integration” will be used to describe that period of time in which the brain is able to process and perceive distinct visual or sound stimuli.

The present invention improves upon known modalities of use of visual flickering and sound to assess brain processing speed.

### SUMMARY OF THE INVENTION

As to visual stimuli, the invention of this application is directed to apparatuses and methods comprising a visual display grid. The display has multiple grid regions each visually displaying a flickering image. The grid regions may be displayed in random locations on a viewing screen of the apparatus. In the alternative, the grid regions may be displayed in an equal number rows and columns or the number of rows and columns may vary. The apparatus of the present invention never displays the same image into all regions of the grid at the same time. That is, at any given instant the actual number or nature of images displayed in grid regions is less than the total number of grid regions in the grid.

The actual or instance number or nature of images simultaneously displayed is selected by the apparatus or by the tester. The apparatus/software does, however, display the image(s) randomly throughout all grid regions so that the image(s) is/are displayed in each grid region at some time. The image may comprise any recognizable and describable shape, color or configuration.

Initially the images are presented at a frequency typically higher than the expected critical flicker fusion threshold of a viewer. As a result, the person viewing the grid perceives that all grid regions present a steady or constant image because the images are presented throughout the grid at a frequency above the anticipated critical flicker fusion threshold of the person. The frequency of the presentation of the images is then decreased until only the instance number and/or nature of images is perceived by the person being analyzed.

The present invention allows the apparatus or the tester to select the instance number and/or nature of the images actually being presented throughout the grid thereby preventing the person being analyzed from merely guessing when a perceived change occurs and what the nature of the perceived change includes. That is, the person being tested must not only indicate when less than all grid regions display an image but must also indicate how many or what type of images appear in the grid regions when a visual change is perceived by the person.

This improvement upon the use of flicker technology to measure brain processing speed has several advantages over the prior art. It allows the device or the tester to affirmatively control the instance number and nature of the images being presented and thereby provides testing reliability as to the measured frequency at which changes in the grid display are perceived by the person being tested. This new flicker technique also allows the device or tester to vary the frequency rate of the display of the images at constant or variable time intervals above, at and below the person's critical flicker fusion threshold. This methodology allows the device or tester to quickly verify the person's critical flicker fusion threshold by changing the instance number and/or nature of the images presented in the grid regions. As a result, in a matter of minutes, the critical flicker fusion threshold and corresponding brain processing speed of the person can be reliably tested and verified.

While the methods and processes of the present invention have proven to be particularly useful in the area of brain speed processing, those skilled in the art can appreciate that the methods and processes can be used in a variety of different applications and in a variety of different areas to aid assessment of diminished or increased brain capacity, brain processing speed of images under the influence of disease, medication, substance abuse, environmental stresses such as light, heat or humidity, fatigue, or under the rigors of workplace requirements and capacities, and the like.

As to sound stimuli, the present invention is directed to apparatuses and methods directed to the selection and broadcasting of a series of commonly utilized sounds such as words at different rates to determine the rate at which the person is able to perceive, that is comprehend, the words. This reveals the rate at which the person's auditory cortex is able to process a series of spoken words thereby permitting the person to comprehend or understand the words.

These and other features and advantages of the present invention will be set forth or will become more fully apparent in the description that follows and in the appended claims. The features and advantages may be realized and obtained by means of the instruments and combinations particularly pointed out in the drawing, descriptions and appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to exemplary embodiments which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates one embodiment of the invention in which an apparatus randomly displays a selected image in a given instance number of grid regions of a viewing screen of the apparatus and at random locations. The images, though not actually displayed simultaneously in each region of the grid, are perceived by the person being tested to be present in each region of the grid because the image is displayed in all regions of the grid in a single window of integration of the person being tested. That is, the images of the entire grid are displayed in an amount and/or at a frequency above the person's critical flicker fusion threshold causing the person to perceive that all regions of the grid display the image at the same time.

FIGS. 2A-2F illustrate examples of other perceived visual displays of the randomized grid regions of FIG. 1 in which the selected image is perceived by the person being tested to be displayed in less than all of the regions of the grid because only one instance of a dual image is displayed in a single window of integration of the person being tested. That is, the instance number of the dual images is displayed at a frequency at or below the person's critical flicker fusion threshold causing the person to perceive that less than all the regions of the grid display the instance number of the dual image. It is contemplated that the color of each instance of the image may be the same. It is also contemplated that when the instance number is greater than one, that each displayed image comprise one or more colors or different colors, thereby providing the tester of additional data against which to verify the person's correct perception of any changes in the grid display.

FIG. 3 illustrates another embodiment of the invention in which a visual grid randomly displays an instance number of the selected images in rows and/or columns. Though not actually displayed simultaneously in each region of the grid, which images are perceived by the person being tested to be present in each region of the grid because more than one instance number of a dual image is displayed in a single window of integration of the person being tested. That is, the images of the entire grid are displayed in an amount and/or at a frequency above the person's critical flicker fusion threshold causing the person to perceive that all regions of the grid display the image.

FIGS. 4A-4F illustrate examples of other perceived visual displays in rows and/or columns of the selected dual image of FIG. 3 in which the same visual grid display of the same selected dual image is perceived by the person being tested to be displayed in less than all of the regions of the grid because only one instance number of the dual image is displayed in a single window of integration of the person being tested. That is, the instance number of the images is displayed at a frequency at or below the person's critical flicker fusion threshold causing the person to perceive that less than all the regions of the grid now display the only the instance number of the image(s).

FIG. 5 illustrates another embodiment of the invention in which a visual grid display of more than one different selected image at random locations, though not actually displayed simultaneously in each region of the grid, is perceived by the person being tested to be displayed in each region of the grid because more than one instance of a dual images is displayed in a single window of integration of the person being tested. That is, the dual image is actually displayed in grid regions in an amount equal to the instance number which is less than the total number of grid regions in the entire grid but at a frequency above the person's critical flicker fusion threshold causing the person to perceive that all regions of the grid display the selected dual image at the same time.

FIGS. 6A-6F illustrate examples of other perceived visual displays of the selected dual image of FIG. 5 in which the same instance number of the selected dual image displayed at random locations of the grid is perceived by the person being tested to be displayed in less than all of the regions of the grid because only one instance number of the images is displayed in a single window of integration of the person being tested. That is, the instance number of the images is displayed at a frequency at or below the person's critical flicker fusion threshold causing the person to perceive that one instance number of the dual image is displayed in less than all the regions of the grid display.

FIG. 7 illustrates another embodiment of the invention in which a column and/or row configuration is used. In this embodiment, the visual grid display of one instance number of different selected images, though not actually displayed simultaneously in each region of the grid, is perceived by the person being tested to be displayed in each region of the grid because more than one instance of the images is displayed in a single window of integration of the person being tested. That is, the images are actually displayed in grid regions in an amount equal to the instance number which is less than the total number of grid regions in the entire grid and at a frequency above the person's critical flicker fusion threshold causing the person to perceive that all regions of the grid display the selected images at the same time.

FIG. 8A-8F illustrate examples of other perceived visual displays of the selected images of FIG. 7 in which the same visual grid display of the same selected images is perceived by the person being tested to be displayed in less than all of the regions of the grid because only one instance of the images is displayed in a single window of integration of the person being tested. That is, the instance number of the images is displayed at a frequency at or below the person's critical flicker fusion threshold causing the person to perceive that less than all the regions of the grid display the images at the same time.

### DETAILED DESCRIPTION OF THE INVENTION

The invention of this application is directed to quickly and reliably identifying the brain processing speed of a person being test. The ability to do so is useful because speed of brain processing is an important factor in many areas such as eye-brain reception and comprehension, reading and reading comprehension, reaction time, mental and physical wellbeing, increasing of brain processing speed, capacity of the person to perform specific tasks such as jobs skills in the workplace, motor reaction time, safety issues and the like.

### Visual Stimuli

As to visual stimuli, the present invention is directed to apparatuses and methods for identifying the rate of visual frequency at which a person perceives distinct images. The apparatuses and methods comprise a screen or projection of visual images configured to visually and as selected randomly display one or more distinct images in a displayed grid configuration. The grid display is viewed by the person to the tested. At different times and under different controlled rate or frequency conditions selected by the device or the tester what the person visually perceives on the grid display appears to change. The identification of these perceived changes by the person provides the device/tester with data to reliably identify and verify the person's window of integration by using flicker fusion principles in a novel and unique way.

The grid display of the present invention can be of any random, geometric or row-and-column configuration. For example, the grid display comprising a constant number of grid regions with the location of the grid regions randomly changing locations on the viewing screen. In another embodiment, the location of the grid regions on the viewing screen may remain static. In another embodiment, the grid display may comprise an equal number of grid regions randomly displayed on the viewing screen. In the alternative, the grid can comprise an equal number of rows and columns such as three-by-three, four-by-four, etc., or the number of rows and columns may vary such as three-by-four, four-by-five, etc. The device or tester may select the nature of the grid display, the number of grid regions and/or number of instances of and the nature of the distinct image or images to be displayed in the regions of the grid. This selection can be done by an algorithm processed by the device or by input from the tester. After these selections are made, the grid display is populated with images.

For example, in a grid randomly displaying seven grid regions, the chosen or instance number of images will be randomly and simultaneously displayed and moved about the viewing screen in less than seven regions. In another embodiment the location of grid regions remains static such as a three-by-three grid with nine grid regions in which the distinct image(s) will only be displayed in less than nine regions of the grid at any given point in time, in a four-by-four grid the distinct image(s) will only be displayed in less than sixteen regions of the grid at any given point in time, and so on. The apparatus/software displays the image(s) randomly throughout all grid regions so that the image(s) is/are presented in each grid region at some time. Preferably, the chosen apparatus or system of the present invention does not broadcast the same image into all regions of the grid at the same time, but it may do so at rates above the person's critical flicker fusion threshold. Preferably, at any given point in time the instance number or nature of images visually presented throughout the grid is in fact displayed in less than the total number of grid regions. For purposes of this application the term “multi-region grid” shall mean a grid comprising either a fixed number of grid regions randomly located for viewing or in a fixed location for viewing, one row and more than one column, more than one row and one column, or more than one row and more than one column. For purposes of this application the term “instance number” means the number of times a distinct stimulus or stimuli is simultaneously presented in the display at any point in time.

Initially the selected images are displayed at a first frequency typically higher than the expected critical flicker fusion threshold of the person viewing the grid display. As a result, the person viewing the grid perceives that all grid regions display steady or constant images in all regions of the grid because the images are displayed throughout the grid at a frequency above the anticipated critical flicker fusion threshold of the person. Stated another way, more than one instance of the selected image(s) is displayed in the person's window of integration.

The frequency of the display of the images is then incrementally decreased until only the actual, selected or instance number and/or nature of images is accurately perceived by the person being tested. At which point the person being tested is able to provide input to the device or some indication to a tester of the perceived change in the display. The perceived change described by the person is compared to the selected conditions to confirm accuracy of the perceived change in the display; the device/tester verifies the instance number and correct identification of the image(s). When verified, the device/tester then captures data as to the second display frequency or rate at which the person correctly perceived the change in the grid display. The captured display frequency can be used by the device or tester in assessing the brain processing speed of the person.

The rate at which the frequency of flicker display is decreased may also vary as to the time interval between decreases.

The process is then repeated by another selection the nature of the grid, the number of grids in the multi-region grid, the nature of the image(s) and the instance number of the image(s). The selected images are displayed at a frequency higher than the previously captured frequency data of the perceived change in the grid display. The display frequency is decreased until the person again correctly perceives a change in the grid display. Again the perceived change is verified and frequency data is captured to identify the frequency of display at the time of the perceived change.

This process is repeated until the device/tester collects enough data to statistically and reliably identify the rate of the display frequency at which the person accurately perceived the change in the grid display. This captured rate of display frequency can then be utilized by the device/tester in assessing the person's speed of processing visual images.

This improved testing technique of the present invention allows the selective control of nature of the grid and the number and/or nature of the images actually being presented throughout the grid thereby preventing the person being analyzed from merely guessing when a perceived change occurs. That is, the person being analyzed must not only indicate when less than all grids contain an image but must also indicate how many and/or what type of image(s) appear in the grid at any given instant at or below the person's flicker fusion threshold.

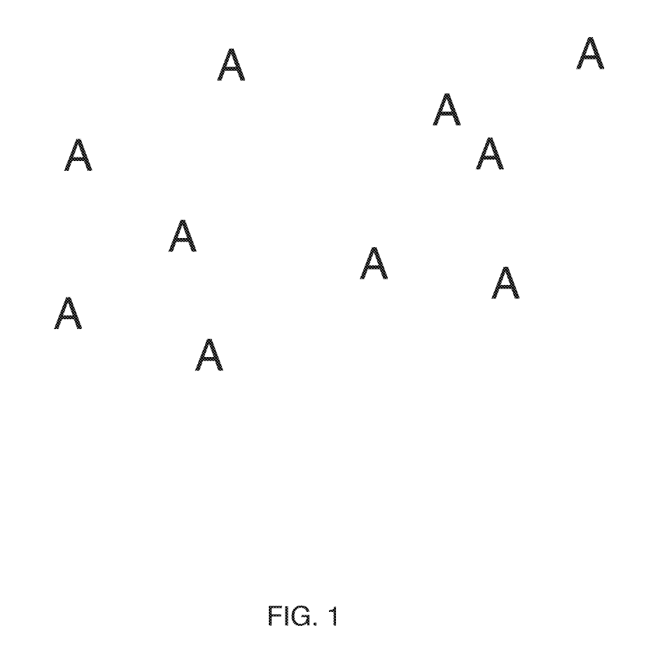

Examples of implementation of the present invention are illustrated in FIGS. 1-8. FIG. 1 illustrates one embodiment of the present invention in which a fixed number of grid regions are selected and displayed in random locations in the grid. As shown in FIG. 1, the fixed number of grid regions of the grid is 10. An image of the letter “A” was selected as the image to be displayed. The instance number of images of “A” to be randomly but simultaneously displayed in grid regions at any given instance was selected as two or a set of “A”s. The apparatus providing or projecting the display randomly displays two images of “A” in the grid regions at an initial or first display frequency sufficiently high so as to be above the anticipated flicker fusion threshold of a typical person. Because the display frequency of “A” is higher than the anticipated flicker fusion threshold of the person being tested multiple images of sets of “A” are displayed in grid regions in one window of integration of the person causing the person to perceive that “A” is simultaneously displayed in up to ten regions of the grid when in fact an image of “A,” unbeknownst to the person being tested, is only displayed in two grid regions of the grid at any given point in time.

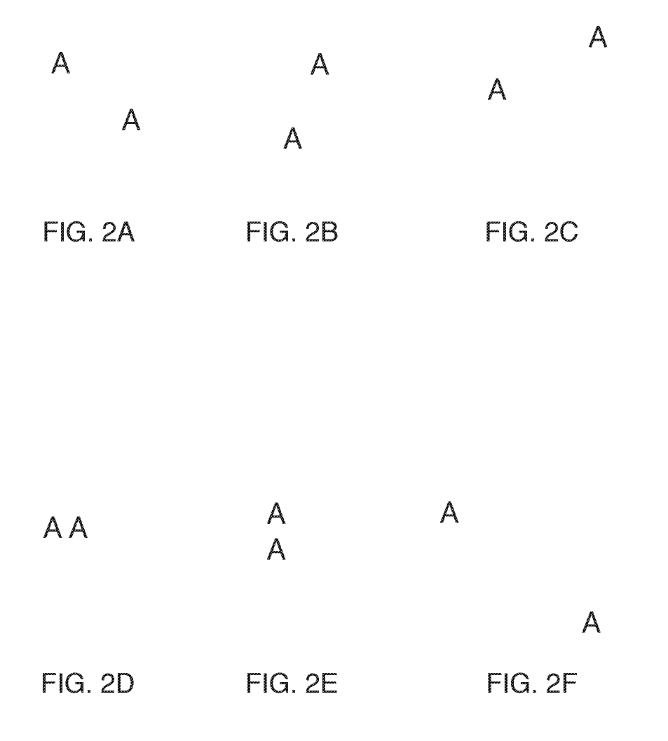

The frequency of the display of the sets of “A” is incrementally decreased until the person being tested perceives a change in the display at a second display frequency. At the point the person perceives a change in the display the second display frequency rate of the sets of “A” is captured. The device/tester confirms by communication from the person being tested how the display changed including identifying how many images of “A” the person sees at any given point in time. If the person accurately sees and communicates seeing correct the instance number, in this example two images, of “A” in the grid regions at any point in time, as shown by example in FIGS. 2A-F, then the device/tester can rely on the captured second display frequency as data from which to calculate the speed at which the person's brain processes visual images.

This technique is repeated by selecting another grid size and number of regions, another image or images to be displayed and the instance number of the image(s) to be displayed at any given point in time. Again starting at the first display frequency or at some display frequency higher than the previously captured or second display frequency, the display frequency is again decreased until the person perceives a change in the grid display consistent with the subsequent selection of image and instance number of images to be displayed at any given point of time. If the person accurately perceives the selected image and number of images, then a second rate of display frequency is again captured and compared to the previously captured display frequency. This process is repeated until the device/tester captures enough samples of second rate of display frequency when the person perceives a change in the display to satisfy statistical data requirements for reliable use the data to analyze or assess the rate at which the person's brain processes visual stimuli.

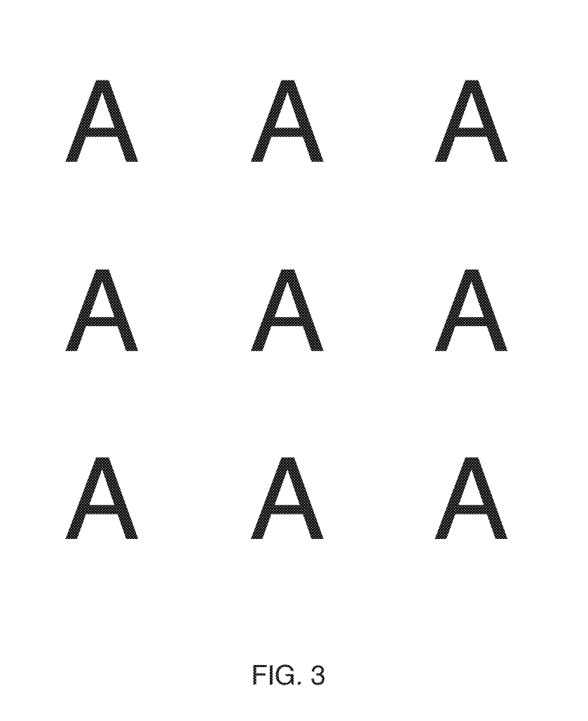

FIG. 3 illustrates another embodiment of the present invention in which a three-by-three, row-and-column grid having nine grid regions was selected. An image of the letter “A” was selected as the image to be displayed. The instance number of images of “A” to be randomly and simultaneously displayed in grid regions at any given instance was selected as two. The apparatus providing the display randomly displays two images of “A” in the grid regions at an initial or first display frequency sufficiently high so as to be above the anticipated flicker fusion threshold of a typical person. Because the first display frequency of display of “A” is higher than the anticipated flicker fusion threshold of the person being tested multiple instances of “A” are displayed in grid regions in one window of integration of the person causing the person to perceive that “A” is simultaneously displayed in more than two but up to nine regions of the grid when in fact an image of “A,” unbeknownst to the person being tested, is only displayed in two grid regions of the grid at any given point in time.

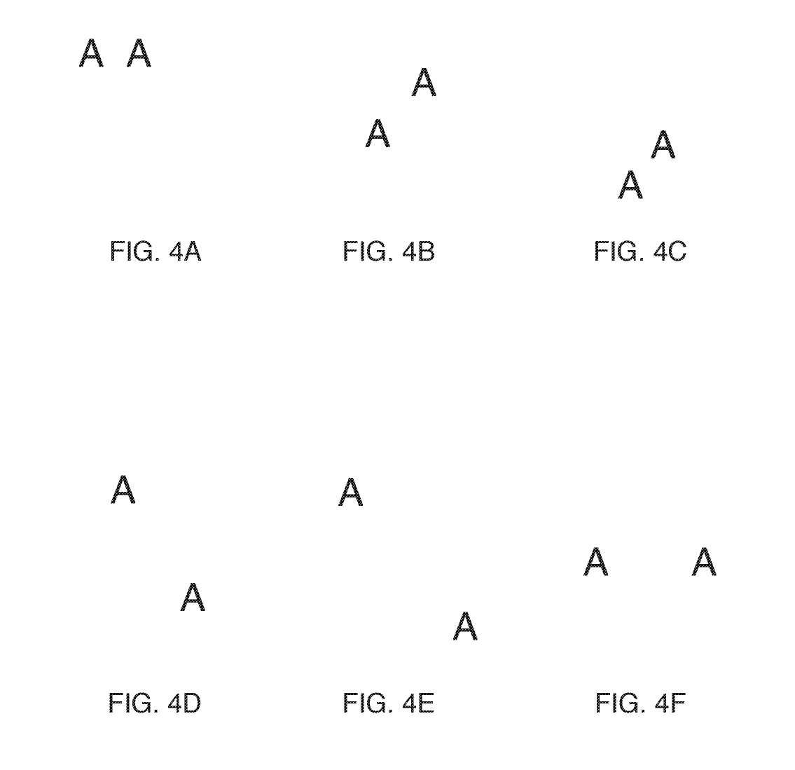

The frequency of the display of the instances of “A” is incrementally decreased until the person being tested perceives a change in the display. At the point the person correctly perceives a change in the display a second display frequency rate of the sets of “A” is captured. The device/tester confirms by communication from the person being tested how the display changed including identifying how many images of “A” the person sees at any given point in time. If the person accurately sees and communicates seeing two images of “A” in the grids at any point in time, as shown by examples in FIGS. 4A-F, then the device/tester can rely on the captured second display frequency as data from which to calculate the speed at which the person's brain processes visual stimuli or images.

This technique is repeated by selecting another grid size, another image to be displayed and the instance number of images to be displayed at any given point in time. Again starting at a first display frequency or at some display frequency higher than the previously captured or second display frequency, the display frequency is again decreased until the person perceives a change in the grid display consistent with the subsequent selection of image and number of images to be displayed at any given point of time. The frequency rate is captured as a second frequency rate. The person confirms the nature of the selected image and instance number of images. This process is repeated until the device/tester captures enough samples of the second rate of display frequency when the person correctly perceives a change in the display to satisfy statistical data requirements for reliable use of the data to analyze or assess the rate at which the person's brain processes visual stimuli.

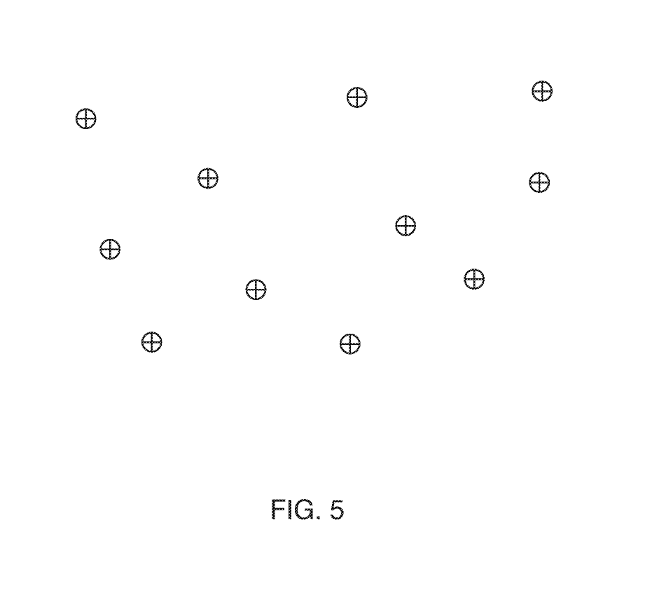

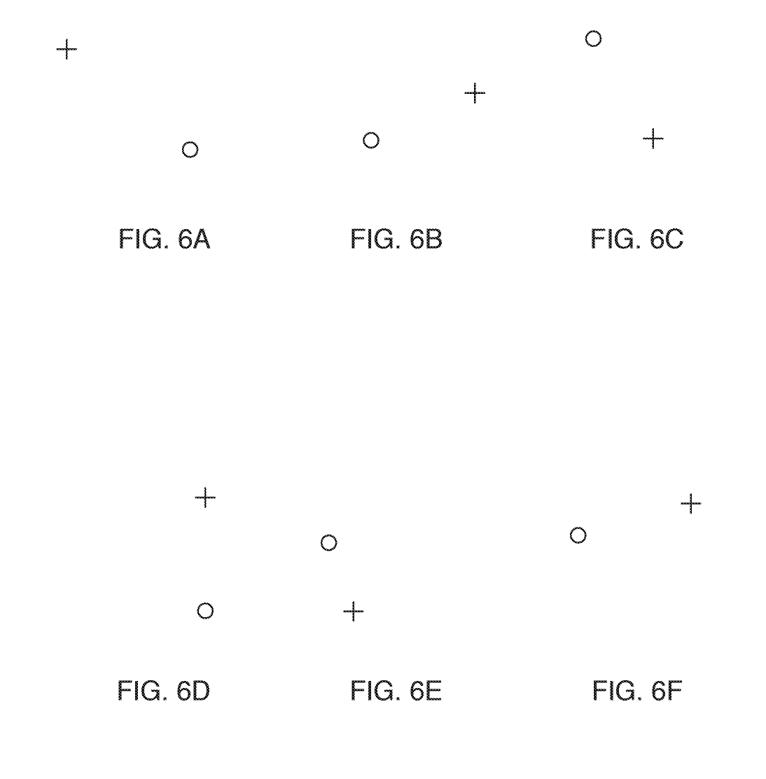

Another exemplary embodiment of the present invention is illustrated in FIGS. 5-6. FIG. 5 illustrates another exemplary embodiment of the present invention in which a fix number of randomly located grid regions is eleven. An image of “O” and an image of “+” were selected as the images to be displayed. The number of instances of both images “O” and “+” to be simultaneously and randomly displayed in grid regions at any given instance was selected as one. The apparatus providing the display randomly and simultaneously displays images “O” and “+” in grid regions at an initial or first display frequency sufficiently high so as to be above the anticipated flicker fusion threshold of a typical person. Because the display frequency of images “O” and “+” is higher than the anticipated flicker fusion threshold of the person being tested multiple images of sets of images “O” and “+” are displayed in grid regions in one window of integration of the person causing the person to perceive that “⊕” is simultaneously displayed in up to all regions of the grid as shown in FIG. 5 when in fact one image of “O” and one image of “+,” unbeknownst to the person being tested, are being only displayed independently and simultaneously in two different grid regions of the grid at any given point in time.

The frequency of the display of images “O” and “+” is incrementally decreased until the person being tested perceives a change in the display at a second display frequency. At the point the person perceives a change in the display the second display frequency rate of the images “O” and “+” is captured. The device/tester confirms by communication from the person being tested how the display changed including identifying how many images of “O” and of “+” the person sees at any given point in time. If the person accurately sees and communicates seeing one image “O” and one image of “+” at any point in time, as shown by example in FIGS. 6A-6F, then the device/tester can rely on the captured second display frequency as data from which to calculate the speed at which the person's brain processes visual images.

This technique is repeated by selecting another grid size, other images to be displayed and the instance number of images to be displayed at any given point in time. Again starting at a first display frequency or at some display frequency higher than the previously captured or second display frequency, the display frequency is again decreased until the person perceives a change in the grid display consistent with the subsequent selection of images and the instance number of each image to be displayed at any given point of time. If the person accurately perceives the selected images and number of images, then the capture rate of display frequency is compared to the previously captured display frequency. This process is repeated until the device/tester captures enough samples of the second rate of display frequency when the person correctly perceives a change in the display to satisfy statistical data requirements for reliable use the data to analyze or assess the rate at which the person's brain processes visual stimuli. It is contemplated that the instance number for “O” and the instance number for “+” may be different.

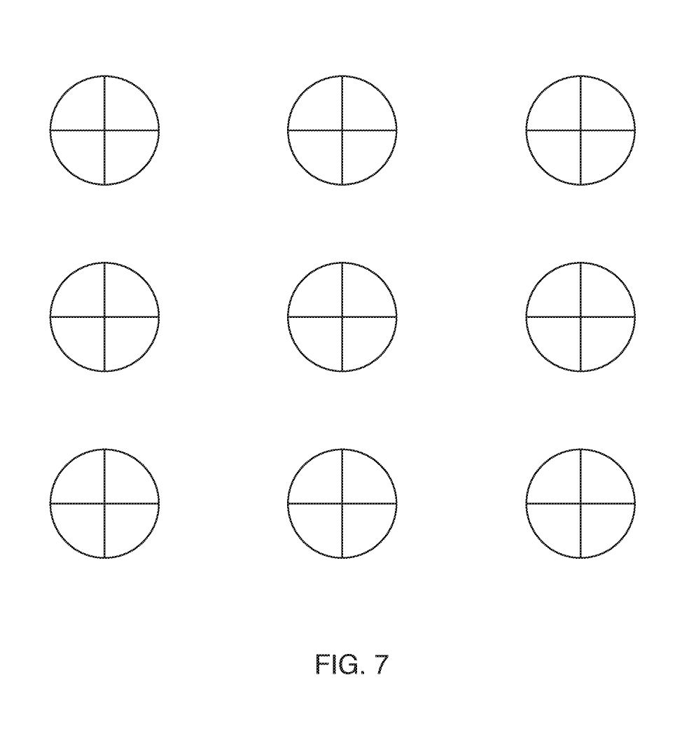

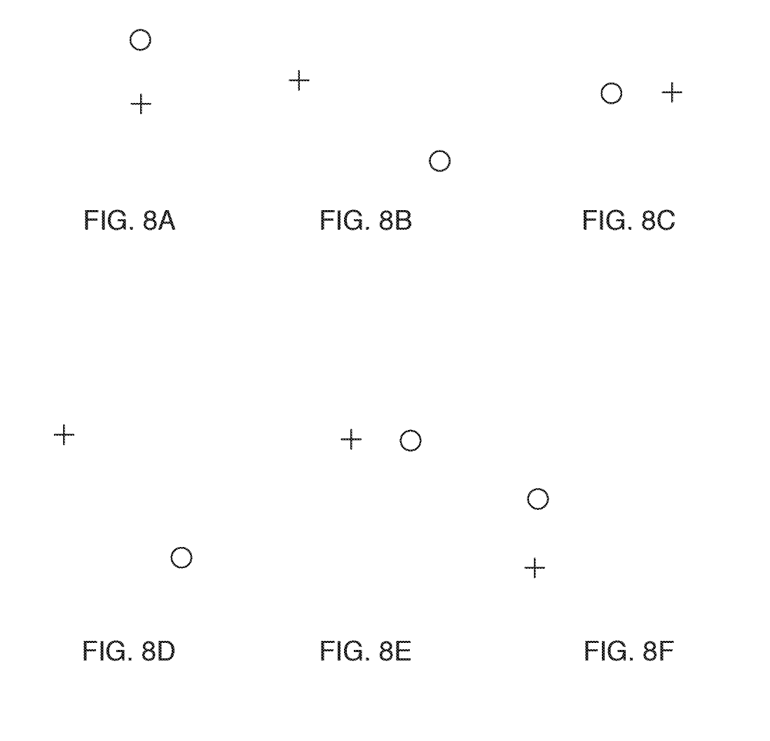

Another exemplary embodiment of the present invention is illustrated in FIGS. 7-8. FIG. 7 illustrates another exemplary embodiment of the present invention in which a three-by-three, row-and-column grid having nine grid regions was selected. An image of “O” and an image of “+” were selected as the images to be displayed. The number of instances of both images “O” and “+” to be simultaneously and randomly displayed in grid regions at any given instance was selected as one. The apparatus providing the display randomly and simultaneously displays one image of “O” and one image of “+” in grid regions at an initial or first display frequency sufficiently high so as to be above the anticipated flicker fusion threshold of a typical person. Because the display frequency of images “O” and “+” is higher than the anticipated flicker fusion threshold of the person being tested multiple images of instances of images “O” and “+” are displayed in grid regions in one window of integration of the person causing the person to perceive that “⊕” is simultaneously displayed in up to nine regions of the grid as shown if FIG. 7 when in fact images of “O” and “+,” unbeknownst to the person being tested, are being displayed independently and simultaneously in only two grid regions of the grid at any given point in time.

The frequency of the display of images “O” and “+” is incrementally decreased until the person being tested perceives a change in the display at a second display frequency. At the point the person perceives a change in the display the second display frequency rate of the images “O” and “+” is captured. The device/tester confirms by communication from the person being tested how the display changed including identifying how many images “O” and “+” the person sees at any given point in time. If the person accurately sees and communicates seeing one image “O” and one image of “+” at any point in time, as shown by example in FIGS. 8A-F, then the device/tester can rely on the captured second display frequency as data from which to calculate the speed at which the person's brain processes visual images.

This technique is repeated by selecting another grid size, other images to be displayed and the instance number of the image(s) to be displayed at any given point in time. Again starting at the first display frequency or at some display frequency higher than the previously captured or second display frequency, the display frequency is again decreased until the person perceives a change in the grid display consistent with the subsequent selection of images and instance number of images to be displayed at any given point of time. If the person accurately perceives the selected images and the instance number of images, then the second rate of display frequency captured is saved and compared to the previously captured second display frequency. This process is repeated until the device/tester captures enough samples of rate of second display frequency when the person perceives a change in the display to satisfy statistical data requirements for reliable use of the data to analyze or assess the rate at which the person's brain processes visual stimuli again, the instance number of “O” and “+” may be different.

One skilled in the art will appreciate that the invention may be practiced by one or more computing devices and in a variety of system configurations, including in a networked configuration. Implementation of the present invention takes place in association with any conventional device capable of visually displaying or projecting a grid of images on a viewer screen or other surface such that the person being tested can readily see the grid display. The invention requires that the device be capable of allowing the device or tester to select the frequency of the flicker display of images, the grid configuration, the nature of the distinct image(s) to be displayed and/or the instance number of the distinct image which is simultaneously displayed in the grid.

For example, a computer program running on compatible hardware can be configured for implementing within a computer system a method for identifying the visual frequency at which a person perceives distinct visual images, the computer program product comprising a computer readable medium for providing computer program code utilized to implement the method. The computer program code or means comprising executable code for implementing the steps for selecting a multi-region grid configuration, for selecting a distinct image or images, for selecting an instance number of image(s), for visually and simultaneously displaying the distinct image(s) in an amount equal to the instance number in the multi-region grid, for displaying the distinct image(s) at a first display frequency at which frequency the person visually perceives the distinct image(s) as being visually displayed in a number of regions of the multi-region grid greater than the instance number, for decreasing the display frequency of the distinct image on the screen to a second display frequency, the second display frequency being the frequency at which the person visually perceives the distinct image(s) in an amount equal to the instance number, and for capturing the value of the second display frequency.

Those skilled in the art will know how to construct apparatuses and construct hardware and/or software capable of such grid and image display and selection. Apparatuses, software and hardware for application of the present invention can be prepared in a variety of ways by those skilled in the art without undue experimentation thereby providing means for selecting a multi-region grid configuration, means for selecting one or more distinct images, means for selecting an instance number and means for selecting the display frequency of the distinct image(s).

Means for selecting a multi-region grid configuration can be a mathematical algorithm which is processed by the device including hardware and software capable of hosting and executing the algorithm in memory or on a chip of a microprocessor or other electronic device capable of hosting and executing the algorithm to generate and display a multi-region grid. In the alternative, means for selecting a multi-region grid configuration can be achieved by the tester or user inputting into the device the grid configuration parameters as to the number of rows and columns and allowing the hardware and/or software of the device to generate and display the corresponding grid. Such means are well-known in the relevant art.

Means for selecting a distinct visual stimuli or image or images can include selecting from a list of predetermined letters, numbers, symbols, colors or any graphic images which can be or are stored in memory or in other storage mechanisms of the device. In the alternative, the device could include a drawing or photo application which the person or tester identifies and/or selects to generate a custom distinct image. Selection of a predetermined letter, number, symbol, color or any other graphic image or custom design can by an algorithm of the device or by selection by the tester or person to be tested. Such means are well-known in the relevant art.

Means for selecting an instance number can be a mathematical algorithm which is processed by the device including hardware and software capable of hosting and executing the algorithm in memory or on a chip of a microprocessor or other electronic device capable of hosting and executing the algorithm to select an instance number. In the alternative, means for selecting an instance number can be achieved by the tester or user selecting and inputting into the device the desired instance number. Such means are well-known in the relevant art.

Means for selecting the display frequency of the distinct image on a screen or elsewhere can be a mathematical algorithm which is processed by the device including hardware and software capable of hosting and executing the algorithm in memory or on a chip of a microprocessor or other electronic device capable of hosting and executing the algorithm to generate and implement a display frequency. In the alternative, means for selecting a display frequency can be achieved by the tester or user inputting into the device the desired display frequency or range of desired display frequencies and allowing the hardware, software and/or user of the device to generate and implement display frequencies consistent with the present invention. Such means are well-known in the relevant art.

This improvement upon the use of flicker technology to measure brain processing speed has several advantages over the prior art. It provides affirmative yet selectable control of the nature of the grid and the number and nature of the images being displayed thereby providing improved testing reliability as to perceived images and the captured frequency rates at which changes in the grid display are accurately perceived by the person being tested. The present invention allows the device/tester to vary the frequency rate of the presentation of the images above, at and below the person's critical flicker fusion threshold. This methodology allows the device/tester to quickly verify the rate of display frequency at which the person experiences flicker fusion of the selected images. As a result, in a matter of minutes, the flicker fusion threshold and corresponding brain processing speed of the person being tested can be reliably assessed and verified by using a simple visual display of observable images wherein at any given point in time all images are being displayed at the same or different display frequency.

### Sound Stimuli

As to sound or auditory stimuli, the present invention is directed to apparatuses and methods for identifying the rate of auditory frequency at which a person perceives distinct sounds. The apparatuses and methods comprise a speaker or other sound emitting device configured to broadcast selected sounds such as words. The broadcast is heard by the person to the tested. At different times and under different controlled frequency conditions selected by the device or the tester what the person hears and perceives or understands changes. The identification of these perceived changes by the person provides the device/tester with data to reliably identify and verify the person's window of sound perception or integration by using varying rates of broadcast frequencies.

The broadcast of the present invention comprises commonly recognized sounds. For example, the broadcast may comprise a plurality of commonly recognized words broadcast one after another, that is, in series. The device or tester may select the instance number and nature of the sounds to be broadcast. This selection of the instance number or nature of the sounds can be done by an algorithm processed by the device or by input from the tester. The number of sounds selected will be referred to in this application as the instance number of sound. After these selections are made, the broadcast is populated with the selected sounds.

For example, in a broadcast the selected sounds will be randomly broadcast in succession. In another embodiment the selected sounds may be repeatedly broadcast in the same order.

Initially the selected sounds are broadcast at a first rate or frequency typically higher than the expected window of integration of the person hearing the broadcast. As a result, the person hearing the broadcast does not perceive distinct sounds because the sounds are broadcast at a rate or frequency above the anticipated window of integration of the person.

The rate or frequency of the broadcast of the sounds is then incrementally decreased until the actual, selected or instance number of distinct sounds is accurately perceived by the person being tested. At which point the person being tested is able to provide input to the device or some indication to a tester of the perceived understanding of the sounds of the broadcast. The device/tester captures data as to the second broadcast frequency at which the person correctly perceived the sounds in the broadcast. The perceived broadcast described by the person is compared to the selected conditions to confirm accuracy of the perceived broadcast. The second broadcast frequency can be used by the device or tested in assessing the brain processing speed of the person.

The rate at which the frequency of sound broadcast is decreased may also vary as to the time interval between decreases.

The process is then repeated by another selection of the instance and nature of the sounds to be broadcast. The selected sounds are broadcast at a rate or frequency higher than the previously captured broadcast frequency of the perceived sounds. The broadcast rate or frequency is decreased until the person again perceives the selected sounds. Again data is captured to identify a second rate or frequency of broadcast at the time the sounds are correctly perceived. Again the nature of the perceived sounds are compared to the selected sounds to verify the correct comprehension of the sounds of the hearer.

This process is repeated until the device/tester collects enough data to statistically and reliably identify the rate or frequency of broadcast at which the person accurately perceived the sounds. This second captured rate or frequency of broadcast can then be utilized by the device/tester in assessing the person's speed of processing audio stimuli.

This improved testing technique of the present invention allows the selective control of nature of the instance number and/or nature of the sounds actually being broadcast thereby preventing the person being analyzed from merely guessing when the sounds are purportedly perceived. That is, the person being analyze must not only indicate when the sounds are correctly perceived but must also indicate how many and/or what sounds/words are broadcast.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. An apparatus for identifying the rate of frequency at which a person perceives distinct stimulus or stimuli, the device comprising:
a. means for selecting one or more distinct stimulus,
b. means for selecting an instance number of the stimulus,
c. a device to present the stimulus to the person being test,
d. means for presenting the distinct stimulus at a first frequency rate at which frequency the person experiences all the stimulus but is unable to perceive each distinct occurrence of each individual stimulus,
e. means for decreasing the presentation frequency of the stimulus to a second display frequency, the second display frequency being the frequency at which the person perceives each distinct stimulus in an amount equal to the instance number, and
f. capturing the value of the second frequency.

2. The apparatus of claim 1 wherein the stimulus comprises a visual image.

3. The apparatus of claim 2 further comprising means for selecting a multi-region grid configuration.

4. The apparatus of claim 3 wherein the device used to present the visual images is a screen or projection configured to visually and simultaneously display the distinct images in an amount equal to the instance number in the multi-region grid.

5. The apparatus of claim 3 wherein the multi-region grid comprises three rows and three columns.

6. The apparatus of claim 2 wherein the distinct image is a letter or number.

7. The apparatus of claim 2 wherein the instance number is more than one.

8. The apparatus of claim 4 further comprises a second distinct image wherein the screen or projection is configured to also visually and simultaneously display the second distinct image in an amount equal to the instance number or a selected second instance number in the multi-region grid, wherein the distinct images are displayed at a first display frequency at which frequency the person visually perceives the distinct images as being visually and simultaneously displayed in a number of regions of the multi-region grid greater than the instance number(s), and wherein the means for decreasing the display frequency decreases the viral display frequency of the first and second images to a second display frequency at which the person is able to perceive the distinct images being visually and independently displayed in a number or regions of the multi-region grid equal to the instance number (s).

9. The apparatus of claim 1 wherein the stimulus comprises an audible sounds, and the number of sounds in the series being equal to the instance number.

10. The apparatus of claim 9 wherein the device used to present the sound is a speaker or sound emitting device configured to audibly present the series of audible sounds such that the person can hear the series of sounds.

11. The apparatus of claim 10 wherein the sound is a spoken word.

12. The apparatus of claim 11 wherein the instance number is two or more than one.

13. A computer program product for implementing within a computer system a method for identifying the frequency rate at which a person perceives one or more distinct stimulus, the computer program product comprising:
a. a computer readable medium for providing computer program code means utilized to implement the method, wherein the computer program code is comprised of executable code for implementing the steps for:
b. selecting one or more distinct stimulus,
c. for selecting an instance number of the stimulus,
d. presenting the distinct stimulus in an amount equal to the instance number,
e. presenting the distinct stimulus to a person a first frequency rate at which frequency the person is unable to perceive each distinct occurrence of individual stimulus equal to the instance number,
f. decreasing the frequency rate of presentation of the stimulus to a second frequency rate, the second frequency rate being the frequency at which the person perceives each distinct occurrence of individual stimulus in an amount equal to the instance number, and
g. capturing the value of the second display frequency.

14. A method for identifying the rate of rate at which a person perceives one or more distinct stimulus, the steps of the method comprising:
a. providing means for selecting one or more distinct stimulus,
b. providing means for selecting an instance number of the stimulus,
c. providing a device to present the stimulus to the person being test,
d. presenting the distinct stimulus at a first frequency or rate at which frequency the person experiences each stimulus but is unable to perceive each distinct occurrence of each stimulus,
e. providing means for decreasing the presentation frequency of the stimuli to a second display frequency, the second display frequency being the frequency at which the person perceives the each distinct occurrence stimuli in an amount equal to the instance number, and
f. capturing the value of the second display frequency.

15. The method of claim 14 wherein the stimulus comprises letter or number.

16. The method of claim 15 wherein the device comprises a visual screen configured or projection of the stimuli to display the stimulus in a multi-region grid configuration.

17. The method of claim 14 wherein the stimulus comprise spoken words.

18. The method of claim 17 wherein the device comprises a speaker configured to sounds the words in series.
